(19) United States
(12) Patent Application Publication
(10) Pub. No.: US 2023/0240288 A1
(43) Pub. Date: Aug. 3, 2023

(54) FRUIT TREE POLLINATION METHOD FOR PREVENTING AND CONTROLLING ERWINIA AMYLOVORA-CAUSED DISEASE

(71) Applicant: ZHEJIANG UNIVERSITY, Hangzhou City (CN)

(72) Inventors: Binggan LOU, Hangzhou City (CN); Pengfei LIU, Hangzhou City (CN); Shenjun GAO, Hangzhou City (CN); Chao SUN, Hangzhou City (CN); Qikang GAO, Hangzhou City (CN)

(73) Assignee: ZHEJIANG UNIVERSITY, Hangzhou City (CN)

(21) Appl. No.: 18/067,670

(22) Filed: Dec. 16, 2022

(30) Foreign Application Priority Data
Jan. 28, 2022 (CN) 202210104632.7

(51) Int. Cl. A01N 25/12; A01H 1/02; A01N 47/18; A01N 63/50; A01P 1/00

(52) U.S. Cl. CPC A01N 25/12; A01H 1/02; A01N 47/18; A01N 63/50; A01P 1/00

(57) ABSTRACT

The present disclosure provides a fruit tree pollination method for preventing and controlling an Erwinia amylovora-caused disease, belonging to the technical field of plant protection and fruit tree cultivation. The method includes: (1) mixing a pollen nutrient solution with a bactericide, and adding refined pollen to obtain a pollen suspension; alternatively, mixing a bactericide powder with the refined pollen and a filler to prepare a mixed powder; where the bactericide is any one or more selected from the group consisting of albendazole, an antibacterial polypeptide, and zhongshengmycin; and (2) conducting pollination on a fruit tree with the pollen suspension or the mixed powder in a flowering stage.

### CROSS REFERENCE TO RELATED APPLICATION

This application claims the benefit and priority of Chinese Patent Application No. 202210104632.7, filed Jan. 28, 2022; the disclosure of which is incorporated by reference herein in its entirety as part of the present application.

### REFERENCE TO SEQUENCE LISTING

A computer readable XML file entitled “Sequence Listing.xml”, that was created on Dec. 16, 2022, with a file size of about 6258 bytes, contains the sequence listing for this application, has been filed with this application, and is hereby incorporated by reference in its entirety.

### TECHNICAL FIELD

The present disclosure relates to the technical field of plant protection and fruit tree cultivation, and in particular relates to a fruit tree pollination method for preventing and controlling an Erwinia amylovora-caused disease.

### BACKGROUND

Pear fire blight is an international quarantine disease with sudden, explosive, and destructive damages. Erwinia amylovora has a wide range of hosts and can infect more than 140 species of plants in 32 genera of Rosaceae. The Erwinia amylovora mainly infects pome fruit plants in Rosaceae such as pear, apple, hawthorn, crabapple, and quince, causing flower rot, fruit rot, and shoot blight showing a burning appearance, and further leading to death of the whole plant. Pear fire blight occurred successively in many countries in North America, Europe, North Africa, the Middle East, and Oceania. In recent years, the pear fire blight has spread to South Korea, Kazakhstan, and Kyrgyzstan in Asia, as well as Xinjiang and Gansu in China. Pear fire blight causes serious harm to pear and apple planting industries, bringing irreparable losses to many countries.

Studies have shown that Erwinia amylovora can easily infect flower organs, such that pear fire blight is most likely to occur during the flowering stage. Spraying during the flowering stage is the most effective measure to control the pear fire blight. However, most of the pesticides with killing and inhibitory effects on pear fire blight have an inhibitory effect on the germination of pear pollen, and may even cause phytotoxicity to petals and leaves. For example, copper hydroxide, basic copper sulfate, oxine-copper, dicopper chloride trihydroxide, thiodiazole copper, thiosen copper, copper abietate, and cuprous oxide and other agents each have a strong killing effect on the pear fire blight, but cannot be used during pear tree growth. Because when being used in the flowering stage, these drugs can significantly reduce the damage of pear fire blight, but can extremely significantly reduce a fruit setting rate and cause phytotoxicity to flowers and young leaves.

In addition, self-pollinated pear trees have a low fruit setting rate. In order to improve the fruit setting rate of pears, there are currently three pollination methods commonly used in production. A first pollination method is that in pear orchards, in addition to the main varieties, other varieties are planted as pollination trees, or pollination branches are grafted, or pollination branches are cut on flowering trees, and the pollination relies on bees and natural wind. Since bees are the main transmission medium of pear fire blight, it is strictly forbidden to release bees in the infected areas, resulting in a significantly reduced yield. A second pollination method is to collect pollen from other pear varieties, and manually spot, shake, spray and brush the pollen using professional equipment to conduct pollination. Since manual pollination is time-consuming, labor-intensive and inefficient, in order to achieve more efficient pollination, a third pollination method has emerged in recent years, namely liquid pollination. The liquid pollination is to mix collected pollen evenly with a nutrient solution, and complete the pollination by drones or sprayers. A premise of the second and third pollination methods is that the pollen must have an activity and is free of Erwinia amylovora.

At present, most of the pollen for artificial assisted pollination is collected and transported in different places, with an increasing risk of carrying Erwinia amylovora. If the pollen cannot be effectively disinfected to eliminate the Erwinia amylovora, the second or third pollination method may cause catastrophic losses in pears. Currently, measures are taken to strengthen the inspection, quarantine, and detection of exported and imported pollen, which are still difficult to implement in actual work due to various reasons such as limited quarantine conditions and detection technologies.

In conclusion, it is a technical problem to be solved urgently by those skilled in the art to develop a method that can effectively kill Erwinia amylovora carried in the pollen, and can reduce an effect of pesticide application on the germination of pollen tubes during a flowering stage.

### SUMMARY

An objective of the present disclosure is to provide a fruit tree pollination method for preventing and controlling an Erwinia amylovora-caused disease. In the present disclosure, a bactericide with safety to a refined pollen activity is applied to the fruit tree pollination, so as to kill potential Erwinia amylovora in the pollen and on the pear trees. The method can effectively pollinate to improve a fruit setting rate, and prevent the occurrence and damage of pear fire blight.

To achieve the above objective, the present disclosure adopts the following technical solutions.

The present disclosure provides a fruit tree pollination method for preventing and controlling an Erwinia amylovora-caused disease, including the following steps:

(1) mixing a pollen nutrient solution with a bactericide, and adding refined pollen to obtain a pollen suspension; alternatively, mixing a bactericide powder with the refined pollen and a filler to prepare a mixed powder; where the bactericide is any one or more selected from the group consisting of albendazole, an antibacterial polypeptide, and zhongshengmycin; and

(2) conducting pollination on a fruit tree with the pollen suspension or the mixed powder in a flowering stage.

The flowering stage is from the beginning of an early flowering stage (20% blooming) to a full flowering stage (80% blooming), during which the pollination is conducted 1 to 2 times. The 20% blooming means that 20% of flowers on a fruit tree bloom, and the 80% blooming means that 80% of the flowers on the fruit tree bloom.

The fruit tree is a Rosaceae fruit tree including but not limited to pear, apple, hawthorn, crabapple, and quince.

In the present disclosure, albendazole, an antibacterial polypeptide, or zhongshengmycin are used as a bactericide mixed with the refined pollen. Indoor and field studies have shown that the three agents have a significant bactericidal activity against the Erwinia amylovora, which can effectively prevent spread of the pathogen and occurrence of the pear fire blight, and have no significant effect on pollen germination and fruit setting of fruit trees.

In the present disclosure, the pollination method can be applied to a situation that the refined pollen carries the Erwinia amylovora and/or the fruit tree has suffered from the pear fire blight. By killing the Erwinia amylovora carried on the pollen and existing on the surface of original tree bodies, the Erwinia amylovora-caused disease is prevented and treated.

Specifically, the pollination method includes liquid pollination and dry pollination.

When using the liquid pollination, the bactericide is prepared into a stock solution with pure water, and the bactericide stock solution is added in a certain proportion, stirred and mixed during preparing a pollen suspension, and then loaded into pollination equipment for spraying pollination.

The bactericide may have a dosage form including an aqueous solution, a suspension, a wettable powder, a soluble powder, a water dispersible granule, a water emulsion, an emulsifiable concentrate, and a soluble concentrate and other common pesticide formulations.

Preferably, the bactericides in the pollen suspension include: 15 mg/L to 60 mg/L of the zhongshengmycin, 56 mg/L to 167 mg/L of the albendazole, and 2 mg/L to 50 mg/L of the antibacterial polypeptide. In the present disclosure, studies have shown that within the above concentration ranges, the Erwinia amylovora carried on the pollen and originally existing in the flower clusters of pear trees can be effectively killed, without affecting the pollen germination and pollen tube length.

Preferably, the pollen nutrient solution includes the following components by mass percentage: 10% to 15% of sucrose, 0.01% to 0.03% of xanthan gum, 0.05% to 0.1% of calcium nitrate, and 0.01% to 0.1% of boric acid.

An amount of the refined pollen is adjusted according to a growth period of the fruit tree and a spraying method, preferably at 6 g/mu to 40 g/mu. If an orchard is a sapling orchard that has just started to bear fruit and subjected to pollination by drones, the pollen is used at 6 g/mu to 10 g/mu; If the orchard is the sapling orchard that has just started to bear fruit and subjected to pollination by an electric sprayer, the pollen is used at 15 g/mu to 20 g/mu; if the orchard is pollinated by the drones in a full bearing period, the pollen is used at 9 g/mu to 12 g/mu; and if the orchard is pollinated by the electric sprayer in the full bearing period, the pollen is used at 20 g/mu to 40 g/mu.

The pollen suspension prepared according to the above conditions is adjusted according to a spraying mode during application; preferably, in step (2), the pollen suspension is sprayed at 2 L/mu to 50 L/mu. The pollination by drones requires 2 L/mu to 50 L/mu of the pollen suspension each time; and the pollination by an electric sprayer requires 30 L/mu to 50 L/mu of the pollen suspension each time.

When using the dry pollination, a bactericide powder is mixed with the refined pollen and a filler in a certain proportion, and pollination is conducted by spotting, shaking, brushing, and spraying.

The bactericide has a dosage form including a powder, a wettable powder, and a soluble powder.

Preferably, the bactericides in the mixed powder include: 5 mg/g to 10 mg/g of the zhongshengmycin, 5 mg/g to 10 mg/g of the albendazole, and 1 mg/g to 20 mg/g of the antibacterial polypeptide.

More preferably, the mixed powder has 1 mg/g to 10 mg/g of the antibacterial polypeptide.

Preferably, the filler is a lycopodium powder. The lycopodium powder is spores from perennial plants of genus Lycopodium, with shape, size, and specific gravity similar to plant pollen; as a pollen filler, the lycopodium powder can be mixed more effectively, avoiding separation of the pollen with the filler during use to cause uneven pollination.

Preferably, the refined pollen and the filler are mixed at a mass ratio of 1:(3-5). More preferably, the refined pollen and the lycopodium powder have a mass ratio of 1:4.

Preferably, in step (2), the pollination is conducted one time at 60% blooming, or conducted one time at 40% blooming and then one time at 80% blooming. Since pear flowers bloom successively, and have a flowering time generally lasting for half a month, the best time for pear pollination is within 3 d after petals open. In order to effectively pollinate as many flowers as possible, thereby saving labor and improving a pollination efficiency, the pollination is generally conducted twice during the flowering stage: one time at 40% blooming and then one time at 80% blooming. The pollination is conducted on cloudy days or in the morning and evening on sunny days to avoid a high temperature period at noon.

The present disclosure has the following beneficial effects:

(1) In the present disclosure, since the fruit tree is most susceptible to infection of the Erwinia amylovora during the flowering stage, a bactericide with safety to an activity of the refined pollen is mixed with the refined pollen, and the bactericide is sprayed while conducting artificial pollination. Therefore, the pollination and the prevention and control of diseases by spraying are integrated, to reduce the number of spraying, to protect the ecological environment, and to reduce a production cost.

(2) In the present disclosure, the bactericides albendazole, an antibacterial polypeptide, and zhongshengmycin can effectively kill the Erwinia amylovora carried on refined pollen, thereby eliminating a risk of occurrence and spread of the pear fire blight caused by pollen-carrying Erwinia amylovora. The bactericides are safe for refined pollen, can effectively improve a fruit setting rate of fruit trees, and allow fruit farmers to increase production and income.

(3) The method of the present disclosure can effectively kill the Erwinia amylovora on a surface of the original tree bodies, form a protective layer for the flower clusters, and prevent the infection of Erwinia amylovora.

(4) The pollination method has a simple operation, a low cost, and easy acceptance by fruit farmers. In addition, the method is an effective supplement to plant inspection and quarantine, which is beneficial to dredging production and circulation links and improving work efficiency.

### BRIEF DESCRIPTION OF THE DRAWINGS

FIG. 1 shows a single colony shape of an Erwinia amylovora strain XJSZ0102 on an NA medium;

FIG. 2 shows pathogenicity test symptoms of the strain XJSZ0102 on pear flower clusters;

FIG. 3 shows pathogenicity test symptoms of the strain XJSZ0102 on shoots of Pyrus betulifolia Bunge;

FIGS. 4A-4B shows PCR amplification results of a specific fragment of the strain, where FIG. 4A is amplification results of a pair of primers AMS3/AMS4c; FIG. 4B is amplification results of a pair of primers pEA29A/pEA29B; Lane M is Marker; Lane 1 is a standard strain ATCC29850 of Erwinia amylovora, Lanes 2 to 6 are XJSZ01, XJSZ02, XJSZ03, XJSZ04, and XJSZ05, respectively; Lane 7 is water; and Lane 8 is a blank lane;

FIG. 5 shows an identification report of matrix-assisted laser desorption ionization coupled to time of flight (MALDI-TOF) mass spectrometry;

FIGS. 6A-6I show influence of an agent with inhibitory effects on the Erwinia amylovora on pollen germination (observed under a microscope at 4×4), where FIG. 6A is treatment with 800 times dilution of 2% kasugamycin; FIG. 6B is treatment with 600 times dilution of 10% albendazole; FIG. 6C is treatment with 800 times dilution of 20% zinc thiazole; FIG. 6D is treatment with 800 times dilution of 3% benziothiazolinone; FIG. 6E is treatment with 800 times dilution of 33.5% oxine-copper; FIG. 6F is treatment with 300 times dilution of 3% zhongshengmycin; FIG. 6G is treatment with 5000 times dilution of 50% antibacterial polypeptide; FIG. 6H is treatment with 2200 times dilution of 46% copper hydroxide; and FIG. 6I is a positive control;

FIGS. 7A-7C show pollination effect using a pollen suspension to protect and control Erwinia amylovora-induced diseases on pear trees in the field in Example 5, where FIG. 7A is a pollination effect on disease-free pear trees of a pollen suspension prepared using the bactericide antibacterial polypeptide and bacteria-carrying pollen; FIG. 7B is a natural pollination effect of the disease-free pear trees; and FIG. 7C is a pollination effect on the disease-free pear trees of a pollen suspension prepared using a no-bactericide pollen nutrient solution and the bacteria-carrying pollen; and

FIGS. 8A-8C show pollination effect using a mixed powder to control Erwinia amylovora-induced diseases on pear trees in the field in Example 7, where FIG. 8A is a pollination effect on disease-free pear trees of a mixed powder prepared using the bactericide antibacterial polypeptide and bacteria-carrying pollen; FIG. 8B is a natural pollination effect of the disease-free pear trees; and FIG. 8C is a pollination effect of a bacteria-carrying pollen powder on the disease-free pear trees.

### DETAILED DESCRIPTION OF THE EMBODIMENTS

The present disclosure is further described below with reference to specific examples. The following embodiments are illustrative of the present disclosure and should not be construed as limiting of the scope of the present disclosure. Modifications or substitutions made to methods, steps or conditions of the present disclosure without departing from the spirit and essence of the present disclosure fall within the scope of the present disclosure.

The test methods used in the following examples are conventional methods unless otherwise specified; the materials and reagents used are commercially available reagents and materials unless otherwise specified.

Test strains: an Erwinia amylovora strain XJSZ0102 was isolated, identified and tested for pathogenicity by Professor Binggan Lou from the Institute of Biotechnology in Zhejiang University, and then stored at ultra-low temperature. Specifically, a process of isolation, identification, and pathogenicity test included the following steps:

### 1. Isolation, Culture, and Identification

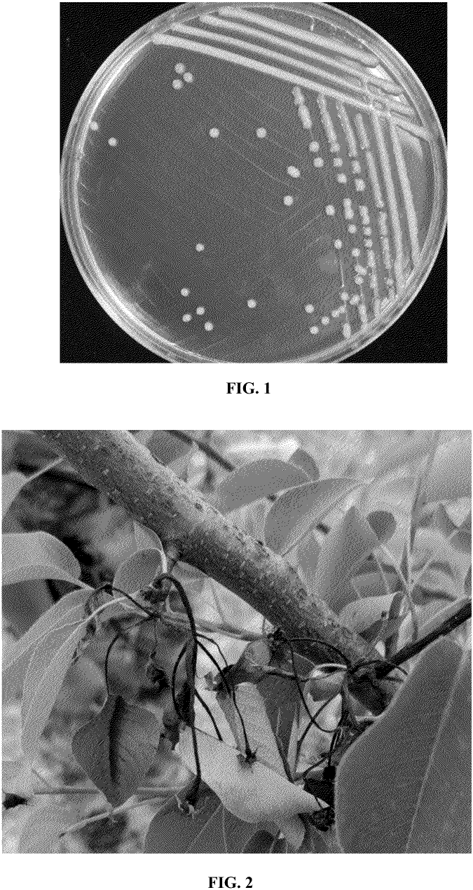

Samples with typical Erwinia amylovora symptoms were collected to prepare a sample suspension, and the sample suspension was streaked and separated on a NA medium plate, where resulting colonies were white, large and convex, and were smooth and lens-like. The colonies were incubated at a constant temperature of 25° C. for 24 h to 48 h, and a suspicious single colony was selected for purification; the purified colony was transferred 3 times, and 5 single colonies were selected and recorded as strains XJSZ0101, XJSZ0102, XJSZ0103, XJSZ0104, and XJSZ0105, where the strain XJSZ0102 had a single colony shape on the NA medium shown in FIG. 1; and the pathogenicity test was conducted.

### 2. Pathogenicity Test

The above 5 strains were streaked on the NA medium separately, and subjected to activated culture for 36 h; a single colony was cultured in an NB medium, and then cultured on a shaker at 28° C. at 150 rpm for 36 h, to prepare a bacterial solution for later use.

### 2.1 Direct-spray Inoculation

The healthy pear flower clusters were selected for direct-spray inoculation until the flower clusters were wet, at an inoculation concentration of 1 ×10⁷ cfu/mL; the incidence was observed and recorded every day; at 5 d to 7 d after the inoculation, each of the 5 strains showed typical Erwinia amylovora symptoms, where the Erwinia amylovora symptoms after infection by the strain XJSZ0102 were shown in FIG. 2.

### 2.2 Acupuncture Inoculation of Seedlings

The healthy pear tree shoots and Pyrus betulifolia Bunge seedling shoots were selected, 20 µL of a bacterial solution was dipped with a sterilized toothpick, and then pricked into inoculated sites at 5 cm away from a top of the shoots, with an inoculation concentration of 1 × 10⁷ cfu/mL. The incidence was observed and recorded every day; at 2 d to 3 d after the inoculation, each of the 5 strains showed typical Erwinia amylovora symptoms, where the strain XJSZ0102 had pathogenicity test symptoms on the Pyrus betulifolia Bunge seedling twigs shown in FIG. 3.

### 3. PCR Identification With Specific Primers

### 3.1 PCR Primer Sequences Were Shown in Table 1

TABLE 1

| | | |
|---|---|---|
| Primer | Sequence | Product |
| AMS3 | 5′-GACGGATCGGCAATCCATTC-3′ | 830 bp |
| AMS4c | 5′-CGCGCATAATTAGCTCT-3′ | |
| pEA29A | 5′-CGGTTTTTAACGCTGGG-3′ | 900 bp |
| pEA29B | 5′-GGGCAAATACTCGGATT-3′ | |

### 3.2 PCR Reaction System and Amplification Conditions

A 25 µL reaction system included: 12.5 µL of a 2× PCR Reaction Master Mix, 1 µL of an forward primer (10 µmol/L), 1 µL of a reverse primer (10 µmol/L), 2 µL of a template, and supplementing with ultrapure water to 25 µL.

For a pair of primers AMS3/AMS4c, a PCR reaction program included: at 95° C. for 3 min; at 94° C. for 30 sec, at 52° C. for 30 sec, and at 72° C. for 1 min, conducting 35 cycles; at 72° C. for 7 min; and storage at 4° C.

For a pair of primers pEA29A/pEA29B, a PCR reaction program included: at 94° C. for 5 min; at 94° C. for 30 sec, at 55° C. for 30 sec, and at 72° C. for 1 min, conducting 35 cycles; at 72° C. for 7 min; and storage at 4° C.

3.3 Results of agarose gel electrophoresis

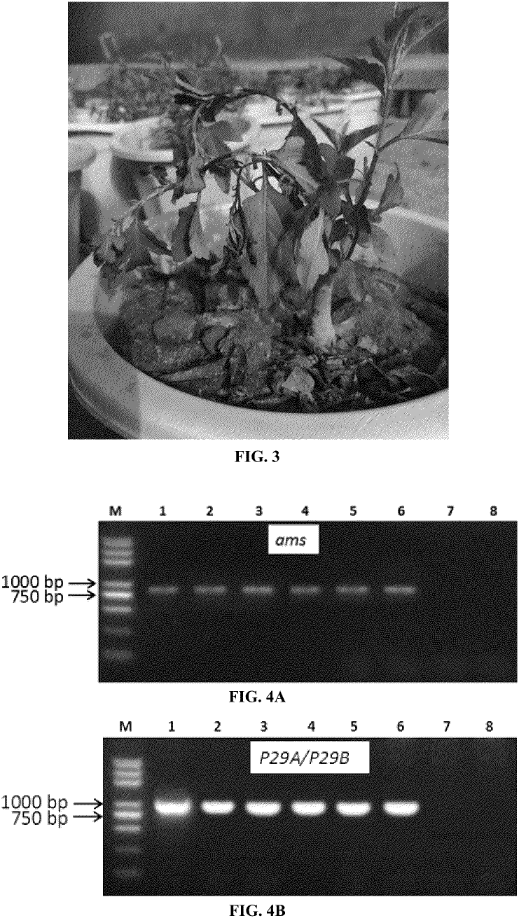

The PCR products were electrophoresed on a 1.5% agarose gel, and then observed and photographed by a gel imaging system. The size of amplification product fragments: the primers AMS3/AMS4c was 830 bp (FIG. 4A), and the primers pEA29A/pEA29B was 900 bp (FIG. 4B), which were consistent with the Erwinia amylovora standard strain ATCC29850.

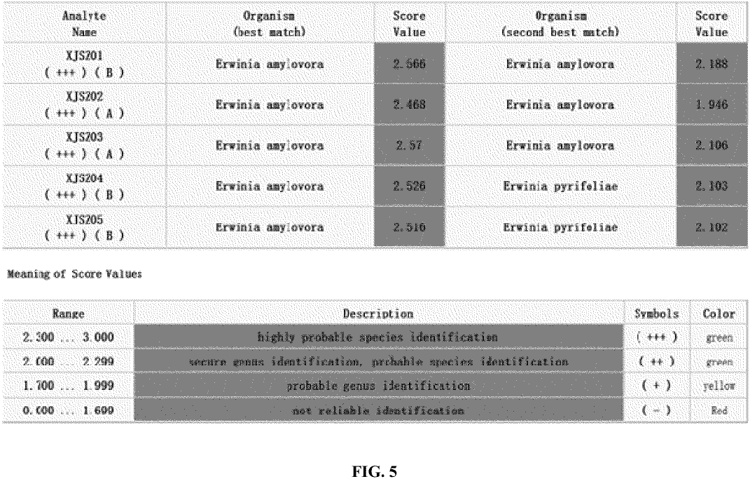

4. The Identification Report of MALDI-TOF Mass Spectrometry Was Shown in FIG. 5. The strain XJSZ0102 was identified to be Erwinia Amylovora

### Example 1 Indoor Toxicity Determination on Inhibition of Erwinia Amylovora by 52 bactericides

### 1. Experimental Materials

Test strain: Erwinia amylovora XJSZ0102.

Test agents: a total of 52 agents were selected for indoor toxicity determination, including antibiotic bactericides, organic copper bactericides, inorganic copper bactericides, compound bactericides, five-membered heterocyclic bactericides, microbial inoculants, enzymic preparations, drugs in development, and other types (Table 2).

TABLE 2

Test agents

| Agent type | SN | Trade name | Active ingredient content/drug name/dosage form | Manufacturer |
|---|---|---|---|---|
| Antibiotic bactericide | 1 | Qunda | 72% agricultural streptomycin sulfate soluble powder | North China Pharmaceutical Group Corporation |
| | 2 | Tetramycin | 0.3% tetramycin aqueous solution | Liaoning Wkioc Bioengineering Co., Ltd. |
| | 3 | Xige | 3% zhongshengmycin aqueous solution | Fujian KLBios Biological Product Co., Ltd. |
| | 4 | / | 2% kasugamycin wettable powder | Qingdao Taisheng Biotechnology Co., Ltd. |
| | 5 | Huanuo Ailei | 6% kasugamycin wettable powder | NCPC Hebei Huanuo Co., Ltd. |
| | 6 | Kasumin | 2% kasugamycin aqueous solution | Jiangmen Plant Protection Co., Ltd. |
| | 7 | Tianchi | 6% kasugamycin wettable powder | Yanbian Kasumin Biological Pharmaceutical Co., Ltd. |
| | 8 | La'ermi | 2% kasugamycin aqueous solution | Shaanxi Tangpusen Biotech Co., Ltd. |
| | 9 | Liangxin | 6% kasugamycin wettable powder | Sinon Chemical (China) Co., Ltd. |
| | 10 | Jundao | 24% validamycin aqueous solution | Wuhan BioKN Co., Ltd. |
| | 11 | Liangye | 8% ningnanmycin aqueous solution | Deqiang Biology Co., Ltd. |
| | 12 | | 88% oxytetracycline HCl wettable powder | Sichuan Jianyang Zhengyang Biochemical Co., Ltd. |
| Organic copper bactericide | 13 | Longpai | 20% thiodiazole copper suspension | Zhejiang Longwan Chemicals Co., Ltd. |
| | 14 | Jingguojing | 33.5% oxine-copper suspension | Sinon Chemical (China) Co., Ltd. |
| | 15 | Lipeiling | 30% thiosen copper suspension | Zhejiang DongFeng Chem. Ind Co., Ltd. |
| | 16 | Tongdao | 12% copper abietate suspension | Guangdong Plant Dragon Biotechnology Co., Ltd. |
| | 17 | Kejunfeng | 30% copper abietate suspension | Liuzhou Huinong Chemical Co., Ltd. |
| Inorganic copper bactericide | 18 | Shangzhicai | 70% dicopper chloride trihydroxide wettable powder | Hunan Shengyu Pharmaceutical Co., Ltd. |
| | 19 | Kocide 3000 | 46% copper hydroxide water dispersible granule | DuPont Corporation, USA |
| | 20 | Tongshifu | 86.2% cuprous oxide wettable powder | Tianjin Lvheng Chemical Co, Ltd. |
| | 21 | Huikepu | 77% copper calcium sulfate wettable powder | English Industries, Mexico |
| | 22 | Cuproxat | 27.2% basic copper sulfate suspension | Nufarm, Australia |
| | 23 | Tianjin | 46% copper hydroxide water dispersible granule | Nufarm, Australia |
| | 24 | Kangxin | 46% copper hydroxide water dispersible granule | Zhejiang Tianfeng Bioscience Co., Ltd. |
| | 25 | Guanjunqing | 57.6% copper hydroxide water dispersible granule | Nufarm, Australia |
| Compound bactericide | 26 | Bimi | 40% kasugamycin·zinc thiazole suspension | Zhejiang Xinnong Chemical Co., Ltd. |
| | 27 | Xingnong Yongfu | 45% kasugamycin·oxine-copper suspension | Sinon Chemical (China) Co., Ltd. |
| | 28 | Fangdajing Tongxi | 36% kasugamycin·oxine-copper suspension | Guangdong Zhenge Biotechnology Co., Ltd. |
| | 29 | Kasumin+Bordeaux | 47% kasugamycin·dicopper chloride trihydroxide wettable powder | Jiangxi Yihe Chemical Co., Ltd. |
| | 30 | / | 5% kasugamycin·zhongshengmycin wettable powder | Shaanxi Biaozheng Crop Science Co., Ltd. |
| | 31 | Amimiaoshou | 32.5% difenoconazole·azoaystrobin suspension | Syngenta Nantong Crops Co., Ltd. |
| | 32 | Jubao | 23% zhongshengmycin·benziothiazolinone suspension | Shaanxi Fengyuan Agricultural Technology Co., Ltd. |
| | 33 | Xizhi | 45% zhongshengmycin·zinc thiazole suspension | Shaanxi Fengyuan Agricultural Technology Co., Ltd. |
| | 34 | Shangge Daliang | 52% dicopper chloride trihydroxide·zineb wettable powder | Shaanxi Shanggezhilu Bioscience Co., Ltd. |
| Five- membered heterocyclic bactericide | 35 | Bisheng | 20%·zinc thiazole suspension | Zhejiang Xinnong Chemical Co., Ltd. |
| | 36 | Xisha | 3%benziothiazolinone wettable powder | Xi'an Hytech Agrochemicals Co., Ltd. |
| | 37 | Xijundi | 10% albendazole suspension | Guizhou Daoyuan Biotechnology Co., Ltd. |
| | 38 | / | 20% bismerthiazol wettable powder | Hubei Qinong Chemical Co., Ltd. |
| Microbial inoculant | 39 | Zhikukang | 1 billion/g bergamot pear Zhikukang microbial inoculant | Henan Bolian Agricultural Research Institute Co., Ltd. |
| | 40 | Xitingfeng | 300 billion/g Pseudomonas fluorescens powder | Xinjiang Fengsuyuan Ecological Agricultural Technology Co., Ltd. |
| Enzymic preparation | 41 | / | KT | CECEP&CIECC Environmental Investment Management Co., Ltd. |
| | 42 | / | MP16 | CECEP&CIECC Environmental Investment Management Co., Ltd. |
| | 43 | / | MP18 | CECEP&CIECC Environmental Investment Management Co., Ltd. |
| | 44 | / | D10 | CECEP&CIECC Environmental Investment Management Co., Ltd. |
| Drugs in development | 45 | / | QD aqueous solution | Xinjiang Shida Wanchuang Technology Information Service Co., Ltd. |
| | 46 | / | QK-1 aqueous solution | Xinjiang Shida Wanchuang Technology Information Service Co., Ltd. |
| | 47 | / | Haishengyuan | Xinjiang Shida Wanchuang Technology Information Service Co., Ltd. |
| | 48 | / | 50% antibacterial polypeptide | such as Funme® peptide Jiangsu Genloci Biotechnologies Inc. |
| Other types | 49 | Tianmengjin | 9% terpene alcohol EC | STK Bio-Technologies, Israel |
| | 50 | Mingsai | 80% sulfur water dispersible granules | Cinobio, USA |
| | 51 | Boqing | 22.7% dithianon suspension | Jiangxi Heyi Chemical Co., Ltd. |
| | 52 | / | German disinfectant | / |

### 2. Experimental Method

### 2.1 Pathogenic Bacteria Culture

The strains preserved in glycerol were streaked on an NA plate and cultured in a biochemical incubator at 28° C. for 48 h; a single colony was selected into an NB medium and incubated in a shaker at 28° C. and 150 rpm for 24 h; an obtained bacterial solution was adjusted to a concentration of 1 × 10⁷ CFU/mL for later use.

### 2.2 Determination by an Inhibition Zone Method

All 52 agents in the test agents were screened with high concentration. 100 µL of a 1 × 10⁷ cfu/mL bacterial suspension was pipetted to spread evenly on an NA plate medium with a diameter of 9 cm. Different agents were prepared into agent solutions each with an active ingredient of 3000 mg/L. A sterilized filter paper with a diameter of 6 mm was fully soaked in the agents, placed on the above streaked plate with a diameter of 9 cm after removing excess liquid; where 3 pieces of paper were evenly placed in each dish, each agent was repeated three times, with sterile water as a control. The plate was incubated in a biochemical incubator at 28° C. for 36 h, and an inhibitory zone diameter (IZD) was measured by a cross method, and statistical analysis was conducted by SPSS software.

### 2.3 Determination by Turbidimetry

A bactericidal effect on Erwinia amylovora was determined by turbidimetry for 19 kinds of agents, including 2% kasugamycin (Kasumin), 40% kasugamycin zinc thiazole, 20% thiazole zinc, 27.2% basic copper sulfate, 20% thiodiazole copper, 46% copper hydroxide (Tianjin), 33.5% oxine-copper, 70% dicopper chloride trihydroxide, 86.2% cuprous oxide, 77% calcium copper sulfate, 57.6% copper hydroxide, 30% thiosen copper, 30% copper abietate, 12% copper abietate, 47% kasugamycin·dicopper chloride trihydroxide, 45% kasugamycin·oxine-copper, 36% kasugamycin·oxine-copper, 52% dicopper chloride trihydroxide·zineb, and 50% antibacterial polypeptide.

A specific method was as follows: a 100 × agent stock solution was prepared according to a recommended use multiple of each agent (dilution factors in Table 3). 500 µL of the agent stock solution was added to 50 ml of a sterilized NB medium, mixed well, and then added with 100 µl of a 1 × 10⁷ CFU/mL bacterial suspension, where each agent was repeated 3 times. A positive control was set with an equal volume of sterile water instead of the agent stock solution, and a negative control was set with an equal volume of sterile water instead of the agent stock solution and the bacterial suspension. After being incubated in a shaking incubator at 28° C. and 150 rpm for 24 h, an OD₆₀₀ increase of each treated culture solution was measured by a UV-Vis spectrophotometer (UV2000, Shanghai Jingke Industrial Co., Ltd.).

$$\begin{matrix}\text{Inhibition} \text{rate =} \\ \frac{\text{Positive control OF increase} \text{} - \text{} \text{Agent treatment OF increase}}{\text{Positive control OF increase}} \times 100 \%\end{matrix}$$

The OD₆₀₀ increase referred to an OD₆₀₀ value of the culture solution subtracting an initial OD₆₀₀ value after culturing for 24 h.

### 3. Results

The results of filter paper method and turbidimetry (Table 3 and Table 4) showed that: among the 52 kinds of agents, agents with an desirable bacteriostatic effect on Erwinia amylovora at a high concentration include: agricultural streptomycin, kasugamycin zinc thiazole, tetramycin, oxytetracycline HCl, benziothiazolinone, albendazole, zhongshengmycin, zinc thiazole, kasugamycin, an antibacterial polypeptide, and most copper preparations. In the subsequent greenhouse experiments of Pyrus betulifolia seedlings and field experiments, it was found that the oxytetracycline HCl and most of the copper preparations had an obvious phytotoxic effect on pear flowers and leaves, while the tetramycin and the benziothiazolinone had a poor protect and control effects on the Erwinia amylovora.

TABLE 3

Antibacterial agents screened by filter paper method and IZDs thereof

| SN | Agent name | IZD (mm) |
|---|---|---|
| 2 | 0.3% tetramycin | 40.2 |
| 12 | 88% oxytetracycline HCl | 33.2 |
| 1 | 72% agricultural streptomycin sulfate | 28.4 |
| 36 | 3% benziothiazolinone | 32.0 |
| 37 | 10% albendazole | 29.4 |
| 3 | 3% zhongshengmycin | 26.5 |
| 30 | 5% kasugamycin·zhongshengmycin | 23.0 |

TABLE 4

Determination results for 18 kinds of agents by turbidimetry

| | | | |
|---|---|---|---|
| SN | Agent name | Dilution factor | Inhibition rate (%) |
| 13 | 20% thiodiazole copper | 500 | 100 |
| 14 | 33.5% oxine-copper | 700 | 100 |
| 29 | 47% kasugamycin·dicopper chloride trihydroxide | 500 | 100 |
| 27 | 45% kasugamycin·oxine-copper | 1200 | 100 |
| 21 | 77% copper calcium sulfate | 500 | 100 |
| 22 | 27.2% basic copper sulfate | 400 | 100 |
| 18 | 70% dicopper chloride trihydroxide | 1000 | 100 |
| 19 | 46% copper hydroxide (Tianjin) | 1500 | 100 |
| 20 | 86.2% cuprous oxide | 800 | 100 |
| 15 | 30% thiosen copper | 900 | 100 |
| 34 | 52% dicopper chloride trihydroxide·zineb | 250 | 100 |
| 28 | 36% kasugamycin·oxine-copper | 2200 | 73.7 |
| 16 | 12% copper abietate | 500 | 58.9 |
| 17 | 30% copper abietate | 1000 | 52.1 |
| 26 | 40% kasugamycin·zinc thiazole | 1000 | 100.0 |
| 35 | 20%·zinc thiazole | 400 | 100.0 |
| 6 | 2% kasugamycin (Kasumin) | 600 | 100.0 |
| 25 | 56.7% copper hydroxide | 2000 | 100 |
| 48 | antibacterial polypeptide | 5000 | 100 |
| | Positive control | | - |

### Example 2 Determination of Safety on Pollen Germination for Agents With Inhibitory Effect on Erwinia Amylovora

### 1. Experimental Materials

Test reagents: the safety on pollen germination was determined on the agents selected in Example 1, including agricultural streptomycin sulfate, oxytetracycline HCl, zhongshengmycin, kasugamycin, antibacterial polypeptide, benziothiazolinone, zinc thiazole, and albendazole, as well as copper hydroxide, basic copper sulfate, and oxine-copper in the copper preparations, as shown in Table 5.

Test pollen: Dangshan pear refined pollen was purchased from the Technical Service Department of Shayidong Gardening Farm in Bayingol Mongolian Autonomous Prefecture, Xinjiang.

### 2. Experimental Method

Preparation of agent stock solutions: a certain amount of each agent was prepared and diluted with distilled water according to a gradient dilution method to obtain the agent stock solutions shown in Table 5.

Preparation of a medicated medium: (1) a 100 mL conical flask was added with 50 mL of pure water and sealed with a disposable sealing film. An obtained solution was boiled in a microwave oven, 10 g of sucrose was immediately added in the conical flask, shaken after sealing to dissolve and continued to heat for boiling; 1 g of an agar powder was immediately added, heated after sealing until the powder was dissolved and a solution became transparent; 0.03 g of boric acid was immediately added, shaken to dissolve for immediate use. (2) 500 µL of the above medium was added to a 2 mL centrifuge tube, added with an equal volume of the agent stock solution, and immediately mixed well by pipetting; 500 µL of a resulting mixture was spread evenly on a glass slide with an area of about 3 cm² and a thickness of about 1 mm, and cooled for later use.

Pollen Activity Detection

the highly active pollen was gently dipped on a cotton ball and blowed normally against the glass slide to scatter the pollen onto the medicated medium. The pollen cultured in a medium prepared with pure water instead of the pesticide was used as a positive control. The culture prepared above was placed in a petri dish with a moist filter paper sheet at the bottom, covered with a lid, and cultivated in a lighted incubator at 25° C. to 28° C. for 4 h. The pollen was observed under an ordinary optical microscope 4×4 or 4×10 (eyepiece×objective), the field of view was moved, the germination status and pollen tube length of more than 100 pollens were counted, and a pollen germination rate was calculated.

$$\text{Pollen germination rate =} \frac{\text{Germinated pollen count}}{\text{Total pollen count}} \times 100 \%$$

### 3. Results

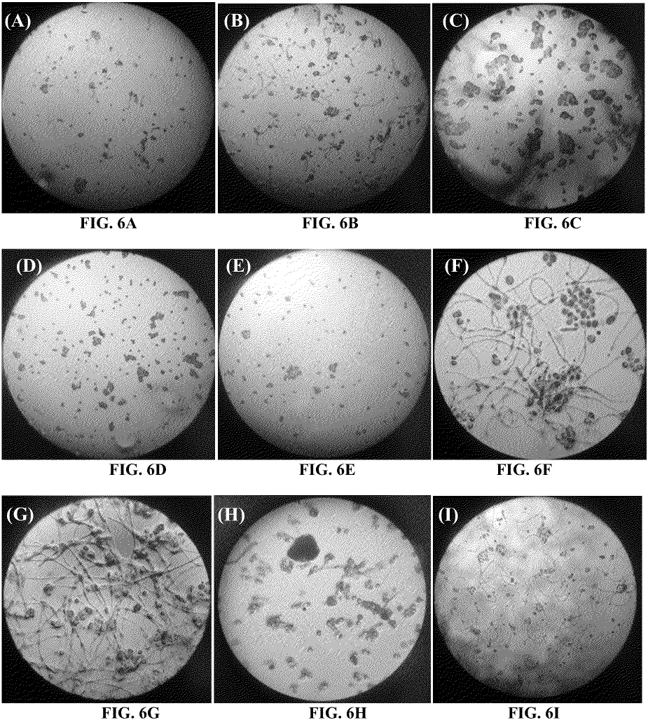

The test results of an effect of 12 agents on the pollen germination rate and pollen tube length were shown in FIGS. 6A-I and Table 5. The results showed that: when the recommended 10% albendazole 600 times dilution, 50% antibacterial polypeptide 5000 times dilution, and 3% zhongshengmycin 500 times dilution were used, the three agents 10% albendazole, 50% antibacterial polypeptide, and 3% zhongshengmycin had no effect on the pollen germination and pollen tube length, which were very safe.

TABLE 5

Test results of effect of 12 bactericides on pollen germination

| Agent name | Dilution factor and effect on pollen germination | | | | | |
|---|---|---|---|---|---|---|
| 72% agricultural streptomycin sulfate | Dilution factor | 250 times | 500 times | 1000 times | 2000 times | 4000 times |
| | Pollen germination rate (%) | 0.00±0.00 | 0.00±0.00 | 0.00±0.00 | 15.37±2.41 | 46.19±3.56 |
| | Pollen tube length∗ | 0 | 0 | 0 | 1/10 | ⅕ |
| 3% zhongshengmycin | Dilution factor | 200 times | 300 times | 500 times | 800 times | 1000 times |
| | Pollen germination rate (%) | 0.96±0.14 | 42.89±1.45 | 64.18±2.17 | 62.59±3.72 | 63.74±2.81 |
| | Pollen tube length∗ | ⅒ | ⅕ | 1 | 1 | 1 |
| 2% kasugamycin | Dilution factor | 150 times | 200 times | 300 times | 500 times | 800 times |
| | Pollen germination rate (%) | 0.00±0.00 | 0.00±0.00 | 3.51±0.37 | 6.40±0.52 | 11.32±0.83 |
| | Pollen tube length∗ | 0 | 0 | 1/10 | ⅒ | ⅕ |
| 88% oxytetracycline HCl | Dilution factor | 250 times | 500 times | 1000 times | 2000 times | 4000 times |
| | Pollen germination rate (%) | 0.00±0.00 | 0.00±0.00 | 0.00±0.00 | 0.00±0.00 | 0.00±0.00 |
| | Pollen tube length∗ | 0 | 0 | 0 | 0 | 0 |
| 33.5% oxine- copper | Dilution factor | 100 times | 200 times | 400 times | 600 times | 800 times |
| | Pollen germination rate (%) | 0.00±0.00 | 0.00±0.00 | 0.00±0.00 | 0.00±0.00 | 0.00±0.00 |
| | Pollen tube length∗ | 0 | 0 | 0 | 0 | 0 |
| 46% copper hydroxide | Dilution factor | 300 times | 600 times | 900 times | 1200 times | 1500 times |
| | Pollen germination rate (%) | 0.00±0.00 | 0.00±0.00 | 0.00±0.00 | 0.00±0.00 | 0.00±0.00 |
| | Pollen tube length∗ | 0 | 0 | 0 | 0 | 0 |
| 27.2% basic copper sulfate | Dilution factor | 75 times | 100 times | 150 times | 250 times | 400 times |
| | Pollen germination rate (%) | 0.00±0.00 | 0.00±0.00 | 0.00±0.00 | 0.00±0.00 | 0.00±0.00 |
| | Pollen tube length∗ | 0 | 0 | 0 | 0 | 0 |
| 56.7% copper hydroxide | Dilution factor | 500 times | 1000 times | 1500 times | 1800 times | 2200 times |
| | Pollen germination rate (%) | 0.00±0.00 | 0.00±0.00 | 0.00±0.00 | 0.00±0.00 | 0.00±0.00 |
| | Pollen tube length∗ | 0 | 0 | 0 | 0 | 0 |
| 20%·zinc thiazole | Dilution factor | 100 times | 200 times | 400 times | 600 times | 800 times |
| | Pollen germination rate (%) | 0.00±0.00 | 0.00±0.00 | 0.00±0.00 | 0.00±0.00 | 0.00±0.00 |
| | Pollen tube length∗ | 0 | 0 | 0 | 0 | 0 |
| 3% benziothiazolinone | Dilution factor | 100 times | 200 times | 400 times | 600 times | 800 times |
| | Pollen germination rate (%) | 0.00±0.00 | 0.00±0.00 | 0.00±0.00 | 0.00±0.00 | 0.00±0.00 |
| | Pollen tube length∗* | 0 | 0 | 0 | 0 | 0 |
| 10% albendazole | Dilution factor | 100 times | 200 times | 400 times | 600 times | 800 times |
| | Pollen germination rate (%) | 0.00±0.00 | 13.39±2.51 | 22.89±1.98 | 62.03±4.18 | 65.28±3.91 |
| | Pollen tube length∗ | 0 | 1/3 | ⅓ | 1 | 1 |
| 50% antibacterial polypeptide | Dilution factor | 5×10³ times | 1×10⁴ times | 2×10⁴ times | 4×10⁴ times | 1×10⁵ times |
| | Pollen germination rate (%) | 62.53±3.17 | 65.48±2.73 | 63.37±3.26 | 62.94±4.19 | 64.72±3.77 |
| | Pollen tube length∗ | 1 | 1 | 1 | 1 | 1 |
| Control (water) | Pollen germination rate (%) | 63.79±4.28 | | | | |
| | Pollen tube length∗ | 1 | | | | |

Note: *the pollen tube length is relative to a pollen tube length of the control group, and the pollen tube length of the control group is set to 1.

### Example 3 Determination of Toxicity of Bactericides Against Erwinia Amylovora

The three pollen-safe agents screened in Example 2 were further studied for their bactericidal activity concentration range.

### 1. Experimental Materials

Erwinia amylovora XJSZ0102.

Test agents:

76.1% albendazole TC (Guizhou Daoyuan Biotechnology Co., Ltd.), 80% zhongshengmycin TC (Fujian KLBios Biological Product Co., Ltd.), and 50% antibacterial polypeptide TC (Jiangsu Genloci Biotechnologies Inc.).

### 2. Experimental Method

Agent dissolution: the albendazole TC was dissolved with glacial acetic acid and diluted with distilled water; the zhongshengmycin TC and the antibacterial polypeptide TC were directly dissolved in distilled water to prepare stock solutions of different concentration gradients.

The NB medium was divided into 50 mL/bottle and sterilized by high-pressure moist heat method, 0.5 ml of the agent stock solution was added to prepare a medicated medium, and 3 bottles were repeated for each concentration. The cultured bacterial solution was adjusted to a bacterial concentration of 1 × 10⁹ cfu/mL, and 50 µL of the bacterial solution was added to each bottle. A bacteria-containing and drug-free medium was used as a positive control. After culturing at 28° C. and 150 rpm for 24 h, an OD₆₀₀ absorbance of the medium in each group was measured; according to survey data, a growth inhibition rate of the bacteria was calculated according to the following equation, expressed as a percentage (%), and the calculation result was rounded to two decimal places.

$$P \left(\%\right) = \left(A 0 - A 1\right) / A 0 \times 100$$

In equation (1):
P stood for the growth inhibition rate;
A0 represented an OD₆₀₀ increase of the positive control; and
A1 represented an OD₆₀₀ increase of the agent treatment.

With SPSS 21.0 software, a toxic regression analysis was conducted on a logarithmic value of the agent concentration and a probability value of the bacteriostasis rate, and a toxic regression curve equation of the bacteriostatic rate of the agent against Erwinia amylovora, an effective inhibition medium concentration EC₅₀ value, and a 95% confidence limit were calculated.

### 3. Results

The results of the toxicity test showed that the albendazole, zhongshengmycin, and antibacterial polypeptide each had a strong killing effect on the Erwinia amylovora and a low EC₅₀ value, with their effective concentrations of 0.022 mg/L, 0.22 mg/L, and 0.12 mg/L, respectively.

TABLE 6

Determination results of toxicity of three kinds of agents against Erwinia amylovora

| Agent | Concentration | Inhibition rate | Regression equation | EC50 | Correlation coefficient (r) | 95% confidence interval (mg/L) |
|---|---|---|---|---|---|---|
| | (mg/L) | (%) | (Y=a+bX) | (mg/L) | | |
| Albendazole | 0.0125 | 15.20 | Y=12.463+4.499X | 0.0220 | 0.9968 | 0.021~0.023 |
| | 0.0175 | 32.97 | | | | |
| | 0.025 | 54.56 | | | | |
| | 0.035 | 81.96 | | | | |
| | 0.050 | 95.36 | | | | |
| Zhongshengmycin | 0.100 | 28.58 | Y=6.1601+1.7404X | 0.2200 | 0.9932 | 0.1904-0.2438 |
| | 0.160 | 40.31 | | | | |
| | 0.256 | 56.63 | | | | |
| | 0.410 | 66.08 | | | | |
| | 0.655 | 81.07 | | | | |
| antibacterial polypeptide | 0.110 | 41.67 | Y=14.285+10.067X | 0.1196 | 0.9899 | 0.11488-0.12448 |
| | 0.133 | 62.25 | | | | |
| | 0.160 | 87.51 | | | | |
| | 0.192 | 98.34 | | | | |
| | 0.230 | 99.82 | | | | |

### Example 4 Indoor Detection of Disinfection Effect of Bactericides on Bacteria-Carrying Pollen

### 1. Experimental Materials

Test agents: 50% antibacterial polypeptide (Jiangsu Genloci Biotechnologies Inc.); 3% zhongshengmycin aqueous solution (trade name Kailikekang, Fujian KLBios Biological Product Co., Ltd.); and 10% albendazole suspension (trade name Xijundi, Guizhou Daoyuan Biotechnology Co., Ltd.).

Test pollen: Dangshan pear refined pollen collected from pear orchards with severe pear fire blight in Halayugong Township, Korla City, Bayingol Mongolian Autonomous Prefecture, Xinjiang, and the pollen was tested to carry Erwinia amylovora.

### 2. Experimental Method

According to concentrations of Table 7, each agent was prepared into an agent working solution with sterile water. 0.1 g of the pollen was added to a 2 mL centrifuge tube, added with 1 mL of the agent working solution, mixed with vortex, and allowed to stand at room temperature for 20 min. 200 µL of a treatment solution was spread evenly on a CCT screening medium with a sterile spreader. The pollen was treated with sterile water and diluted by 1000 times, and 200 µL of a dilution was spread on the CCT screening medium to set as a positive control group, while a sterile water-coated plate was set as a negative control group. The pollen was cultured in a constant temperature incubator at 28° C. for 48 h. The colony growth was observed.

TABLE 7

Concentrations of indoor pollen disinfectants

| | | | |
|---|---|---|---|
| Agent | Dilution factor (concentration) | | |
| 3% zhongshengmycin | 500 times (60 mg/L) | 1000 times (30 mg/L) | 2000 times (15 mg/L) |
| 10% albendazole | 600 times (167 mg/L) | 1200 times (83 mg/L) | 1800 times (56 mg/L) |
| 50% antibacterial polypeptide | 5000 times (50 mg/L) | 25,000 times (10 mg/L) | 125,000 times (2 mg/L) |

Note: values in parentheses were converted into effective concentrations of the agents.

### 3. Results

The test results showed that the zhongshengmycin had an effective concentration of 15 mg/L to 60 mg/L, the albendazole had an effective concentration of 56 mg/L to 167 mg/L, and the antibacterial polypeptide had an effective concentration of 2 mg/L to 50 mg. /L, that is, pathogenic bacteria on the pollen were completely killed within a safe use range of the pollen (Table 8).

TABLE 8

Test results of indoor pollen disinfection

| 3% zhongshengmycin | Agent concentration | 500 times | 1000 times | 2000 times | Positive control | Negative control |
|---|---|---|---|---|---|---|
| | Test results | - | - | - | + | - |
| 10% albendazole | Agent concentration | 600 times | 1200 times | 1800 times | Positive control | Negative control |
| | Test results | - | - | - | + | - |
| 50% antibacterial polypeptide | Agent concentration | 5000 times | 25,000 times | 125,000 times | Positive control | Negative control |
| | Test results | - | - | - | + | - |

Note: “-” meant that no Erwinia amylovora was detected, and “+” meant that the Erwinia amylovora was detected.

### Example 5 Orchard Pollination Test (i) Using Pollination Technology Capable of Blocking Pollen Transmission of Erwinia Amylovora

### 1. Experimental Materials

Test agents: 50% antibacterial polypeptide (Jiangsu Genloci Biotechnologies Inc.); 3% zhongshengmycin aqueous solution (trade name Kailikekang, Fujian KLBios Biological Product Co., Ltd.); and 10% albendazole suspension (trade name Xijundi, Guizhou Daoyuan Biotechnology Co., Ltd.).

Test pollen: Dangshan pear refined pollen collected from pear orchards with severe pear fire blight in Halayugong Township, Korla City, Bayingol Mongolian Autonomous Prefecture, Xinjiang, and the pollen was tested to carry Erwinia amylovora.

Test orchard: the orchard was located in a third branch of Avati Farm, Korla City, Bayingol Mongolian Autonomous Prefecture, Xinjiang; the fruit trees were 8-year-old bergamot pear trees, and the orchard had never experienced pear fire blight.

Test period: April 5 to May 30, 2021

### 2. Experimental Method

10 g of xanthan gum was dissolved in 2.5 L of continuously-boiling water and cooled to obtain a solution I; 6.5 kg of white sugar was dissolved with 10 L of hot water to obtain a solution II; 25 g of calcium nitrate and 5 g of boric acid were dissolved in 0.5 L of water to obtain a solution III; the above three solutions were poured into a large container, then added with 30 L of water, and stirred well to prepare a nutrient solution for later use.

Each agent was diluted with water to form a stock solution, and 100 mL of the stock solution was added to 9.9 L of the nutrient solution, fully stirred and mixed, such that a final concentration of the agent in the nutrient solution was shown in Table 7, and 9 groups of agent treatments were obtained in total.

4 g of the pollen was added to 4 L of a prepared nutrient solution containing the agent, stirred to make the pollen evenly dispersed to prepare a pollen suspension, which was then pollinated by spraying with a shoulder-back electric sprayer. The pollination period was at 40% blooming and at 80% blooming separately, each tree was sprayed with 1 L of the pollen suspension, and each agent treatment group was sprayed to pollinate 3 trees. A pollen suspension prepared with water instead of the agent was set as a positive control, and a pollen suspension prepared with water instead of the pollen was set as a negative control, and 3 trees were treated by each group. 2 weeks after pollination, a fruit setting rate and the number of flower rot and diseased fruit were counted to calculate an incidence.

$$\begin{matrix}\text{Incidence =} \\ \frac{\left(\text{Number of flower rots + number of fruit rots}\right)}{\text{Total number}} \times 100 \%\end{matrix}$$

### 3. Results

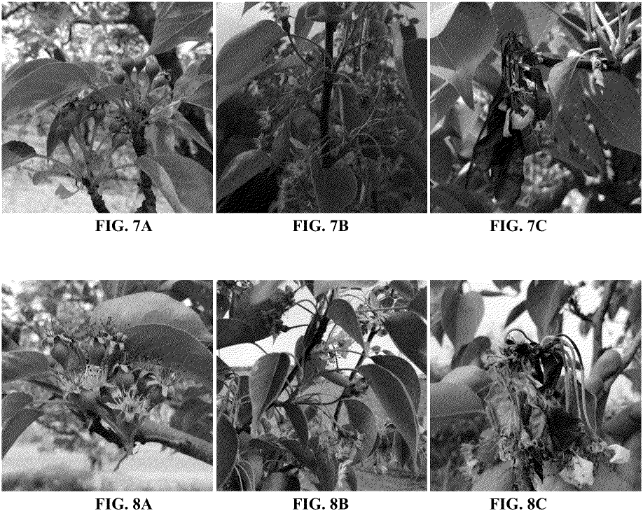

The test results showed (Table 9 and FIGS. 7A-C) that: the liquid pollination technology proposed by the present disclosure to block the pollen transmission of Erwinia amylovora had no significant effect on the fruit setting rate of fragrant pear. Pear fire blight had never occurred in this experimental orchard before. If the artificially-pollinated pollen did not contain Erwinia amylovora, the pear fire blight this year had an extremely low probability in occurrence, just like the negative control area. However, the flower rot and fruit rot caused by pear fire blight had a high incidence in the positive control area. After artificial pollination with pollen suspension containing albendazole, zhongshengmycin, and antibacterial polypeptide separately, the flower rot and fruit rot caused by pear fire blight had a significantly lower incidence than that of the positive control. This indicated that the technology could effectively kill the Erwinia amylovora in pollen to prevent the occurrence of pear fire blight transmitted by the pollen.

TABLE 9

Liquid pollination effect by pear fire blight blocking technology in the field

| 3% zhongshengmycin | Agent concentration | 500 times | 1000 times | 2000 times | Positive control | Negative control |
|---|---|---|---|---|---|---|
| | Fruit setting rate (%) | 72.47±3.27 | 74.96±4.85 | 73.58±3.91 | 76.43±5.29 | 25.31±3.17 |
| | Incidence (%) | 0.00±0.00 | 0.33±0.58 | 1.12±0.22 | 50.52±0.38 | 0.00±0.00 |
| 10% albendazole | Agent concentration | 600 times | 1200 times | 1800 times | Positive control | Negative control |
| | Fruit setting rate (%) | 69.83±5.11 | 72.57±3.16 | 74.16±7.24 | 76.43±5.29 | 25.31±3.17 |
| | Incidence (%) | 0.00±0.00 | 0.00±0.00 | 0.92±0.13 | 50.52±0.38 | 0.00±0.00 |
| 50% antibacterial polypeptide | Agent concentration | 5000 times | 25,000 times | 125,000 times | Positive control | Negative control |
| | Fruit setting rate (%) | 74.82±6.33 | 77.03±6.14 | 75.72±4.89 | 76.43±5.29 | 25.31±3.17 |
| | Incidence (%) | 0.00±0.00 | 0.00±0.00 | 0.00±0.00 | 50.52±0.38 | 0.00±0.00 |

### Example 6 Orchard Pollination Test (II) Using Pollination Technology Capable of Blocking Pollen Transmission of Erwinia Amylovora

### 1. Experimental Materials

Test agents: 50% antibacterial polypeptide (Jiangsu Genloci Biotechnologies Inc.); 3% zhongshengmycin aqueous solution (trade name Kailikekang, Fujian KLBios Biological Product Co., Ltd.); and 10% albendazole suspension (trade name Xijundi, Guizhou Daoyuan Biotechnology Co., Ltd.).

Test pollen: Dangshan pear refined pollen was purchased from the Technical Service Department of Shayidong Gardening Farm in Bayingol Mongolian Autonomous Prefecture, Xinjiang, which was not detected to carry the Erwinia amylovora by the Korla Bergamot Pear Research Center.

Test orchard: the orchard was located in a fourth branch of Avati Farm, Korla City, Bayingol Mongolian Autonomous Prefecture, Xinjiang; the fruit trees were 10-year-old fragrant pear trees, and the orchard was a moderate incidence area of the pear fire blight.

Test period: April 4 to May 30, 2021

### 2. Experimental Method

10 g of xanthan gum was dissolved in 2.5 L of continuously-boiling water and cooled to obtain a solution I; 6.5 kg of white sugar was dissolved with 10 L of hot water to obtain a solution II; 25 g of calcium nitrate and 5 g of boric acid were dissolved in 0.5 L of water to obtain a solution III; the above three solutions were poured into a large container, then added with 30 L of water, and stirred well to prepare a nutrient solution for later use.

Each agent was diluted with water to form a stock solution, and 100 mL of the stock solution was added to 9.9 L of the nutrient solution, fully stirred and mixed, such that a final concentration of the agent in the nutrient solution was shown in Table 7, and 9 groups of agent treatments were obtained in total.

4 g of the pollen was added to 4 L of a prepared nutrient solution containing the agent, stirred to make the pollen evenly dispersed to prepare a pollen suspension, which was then pollinated by spraying with a shoulder-back electric sprayer. The pollination period was at 40% blooming and at 80% blooming separately, each tree was sprayed with 1 L of the pollen suspension, and each agent treatment group was sprayed to pollinate 3 trees. A pollen suspension prepared with water instead of the agent was set as a positive control group, and the pollen suspension prepared with water instead of the agent and sprayed on disease-free trees was set as a negative control group, and 3 trees were treated by each group.2 weeks after pollination, a fruit setting rate and the number of flower rot and diseased fruit were counted to calculate an incidence.

$$\begin{matrix}\text{Incidence =} \\ \frac{\left(\text{Number of flower rots + number of fruit rots}\right)}{\text{Total number}} \times 100 \%\end{matrix}$$

### 3. Results

In this experiment, the pollen did not carry the Erwinia amylovora, but the test orchard was a moderate occurrence area of the pear fire blight. The incidence of pear fire blight-based flower rot and fruit rot was significantly lower than that of the positive control group after conducting artificial pollination with the pollen containing albendazole, zhongshengmycin, and antibacterial polypeptide separately. This indicated that the technology could also kill the original Erwinia amylovora in flower clusters of the pear trees (Table 10).

TABLE 10

Liquid pollination effect by pear fire blight blocking technology in the field

| 3% zhongshengmycin | Agent concentration | 500 times | 1000 times | 2000 times | Positive control | Negative control |
|---|---|---|---|---|---|---|
| | Fruit setting rate (%) | 65.62+4.2 3 | 64.114.72 | 67.14±4.99 | 68.25±2.53 | 8.12±1.93 |
| | Incidence (%) | 0.00±0.00 | 0.00±0.00 | 0.26±0.03 | 43.78±4.26 | 0.00±0.00 |
| 10% albendazole | Agent concentration | 600 times | 1200 times | 1800 times | Positive control | Negative control |
| | Fruit setting rate (%) | 62.54±6.4 7 | 67.81±4.26 | 69.13±6.25 | 68.25±2.53 | 8.12±1.93 |
| | Incidence (%) | 0.00±0.00 | 0.00±0.00 | 0.18±0.05 | 43.78±4.26 | 0.00±0.00 |
| 50% antibacterial polypeptide | Agent concentration | 5000 times | 25.000 times | 125.000 times | Positive control | Negative control |
| | Fruit setting rate (%) | 66.44±1.7 2 | 64.29±5.11 | 67.96±2.56 | 68.25±2.53 | 8.12±1.93 |
| | Incidence (%) | 0.00±0.00 | 0.00±0.00 | 0.00±0.00 | 43.78±4.26 | 0.00±0.00 |

### Example 7 Orchard Pollination Test (III) Using Pollination Technology Capable of Blocking Pollen Transmission of Erwinia Amylovora

### 1. Experimental Materials

Test agent: 50% antibacterial polypeptide (Jiangsu Genloci Biotechnologies Inc.)

Test pollen: Dangshan pear refined pollen collected from pear orchards with severe pear fire blight in Halayugong Township, Korla City, Bazhou, Xinjiang, and the pollen was tested to carry Erwinia amylovora.

Test orchard: the orchard was located in a third branch of Avati Farm, Korla City, Bayingol Mongolian Autonomous Prefecture, Xinjiang; the fruit trees were 8-year-old fragrant pear trees, and the orchard had never experienced pear fire blight.

Test period: April 5 to May 30, 2021

### 2. Experimental Method

The antibacterial polypeptide was mixed with 80 g of a lycopodium powder and 20 g of refined pollen by stirring, to obtain pollen mixtures containing 1 mg/g, 5 mg/g, and 10 mg/g of an active ingredient of the antibacterial polypeptide, respectively; each of the pollen mixtures was dipped with a brush, and brushed slightly on newly-opened flowers with pink anther to complete pollination; while pollination with an agent-free pollen mixture was used as a positive control, and pollination with the lycopodium powder was used as a negative control. Each group had three repetitions, and each repetition had 100 to 120 clusters of flowers. 2 weeks after pollination, the fruit setting rate and the incidence were counted.

### 3. Results

The test results showed that: the solid pollination technology proposed by the present disclosure to block the pollen transmission of Erwinia amylovora had no significant effect on the fruit setting rate of fragrant pear. The method of the present disclosure could kill most of the Erwinia amylovora carried by pollen, thereby significantly reducing the occurrence of pear fire blight in pollinated orchards (Table 11 and FIGS. 8A-C).

TABLE 11

Solid pollination effect of pear fire blight blocking technology in field (antibacterial polypeptide)

| Agent content | 1 mg/g | 5 mg/g | 10 mg/g | Positive control | Negative control |
|---|---|---|---|---|---|
| Fruit setting rate (%) | 88.19±4.97 | 86.36±3.98 | 84.75±5.51 | 87.41±6.45 | 5.31±2.73 |
| Incidence (%) | 4.73±1.94 | 1.45±0.97 | 0.00±0.00 | 79.63±7.86 | 0.00±0.00 |

The foregoing examples are the preferred examples of the present disclosure and are not intended to limit the protection scope of the present disclosure. Any modifications, equivalent substitutions, improvements, etc. made within the spirit and scope of the present disclosure should be included within the protection scope of the present disclosure.

What is claimed is:

1. A fruit tree pollination method for preventing and controlling an Erwinia amylovora-caused disease, comprising the following steps:
(1) mixing a pollen nutrient solution with a bactericide, and adding refined pollen to obtain a pollen suspension; alternatively, mixing a bactericide powder with the refined pollen and a filler to prepare a mixed powder; wherein the bactericide is any one selected from the group consisting of albendazole, an antibacterial polypeptide, and zhongshengmycin, wherein the bactericides in the pollen suspension comprise: 15 mg/L to 60 mg/L of the zhongshengmycin, 56 mg/L to 167 mg/L of the albendazole, and 2 mg/L to 50 mg/L of the antibacterial polypeptide; and the bactericides in the mixed powder comprise: 5 mg/g to 10 mg/g of the zhongshengmycin, 5 mg/g to 10 mg/g of the albendazole, and 1 mg/g to 10 mg/g of the antibacterial polypeptide; and
(2) conducting pollination on a fruit tree with the pollen suspension or the mixed powder in a flowering stage.

2. The fruit tree pollination method for preventing and controlling an Erwinia amylovora-caused disease according to claim 1, wherein in step (2), the pollen suspension is sprayed at 2 L/mu to 50 L/mu.

3. The fruit tree pollination method for preventing and controlling an Erwinia amylovora-caused disease according to claim 1, wherein the refined pollen is applied at 6 g/mu to 40 g/mu.

4. The fruit tree pollination method for preventing and controlling an Erwinia amylovora-caused disease according to claim 1, wherein the pollen nutrient solution comprises the following components by mass percentage: 10% to 15% of sucrose, 0.01% to 0.03% of xanthan gum, 0.05% to 0.1% of calcium nitrate, and 0.01% to 0.1% of boric acid.

5. The fruit tree pollination method for preventing and controlling an Erwinia amylovora-caused disease according to claim 1, wherein the filler is a lycopodium powder.

6. The fruit tree pollination method for preventing and controlling an Erwinia amylovora-caused disease according to claim 1, wherein the refined pollen and the filler are mixed at a mass ratio of 1: (3-5).

7. The fruit tree pollination method for preventing and controlling an Erwinia amylovora-caused disease according to claim 5, wherein the refined pollen and the filler are mixed at a mass ratio of 1: (3-5).

8. The fruit tree pollination method for preventing and controlling an Erwinia amylovora-caused disease according to claim 1, wherein in step (2), the pollination is conducted 1 to 2 times in the flowering stage; specifically, the pollination is conducted one time at 60% blooming, or conducted one time at 40% blooming and then one time at 80% blooming.

9. The fruit tree pollination method for preventing and controlling an Erwinia amylovora-caused disease according to claim 1, wherein the fruit tree is a Rosaceae fruit tree.

10. The fruit tree pollination method for preventing and controlling an Erwinia amylovora-caused disease according to claim 9, wherein the Rosaceae fruit tree is selected from the group consisting of pear, apple, hawthorn, crabapple, and quince.


SEQUENCE LISTING (4 sequences)

SEQ ID NO: 1 - DNA, 20 bp, synthetic construct
  source 1..20: PCR_primers = fwd_name:AMS3,fwd_seq: gacggatcggcaatccattc, rev_name:AMS4c,rev_seq:cgcgcataattagctct; mol_type = other DNA; note = primer AMS3
gacggatcggcaatccattc

SEQ ID NO: 2 - DNA, 17 bp, synthetic construct
  source 1..17: PCR_primers = fwd_name:AMS3,fwd_seq: gacggatcggcaatccattc, rev_name:AMS4c,rev_seq:cgcgcataattagctct; mol_type = other DNA; note = Primer AMS4c
cgcgcataattagctct

SEQ ID NO: 3 - DNA, 17 bp, synthetic construct
  source 1..17: PCR_primers = fwd_name:pEA29A,fwd_seq: cggtttttaacgctggg,r ev_name:pEA29B,rev_seq:gggcaaatactcggatt; mol_type = other DNA; note = Primer pEA29A
cggtttttaacgctggg

SEQ ID NO: 4 - DNA, 17 bp, synthetic construct
  source 1..17: PCR_primers = fwd_name:pEA29A,fwd_seq: cggtttttaacgctggg,r ev_name:pEA29B,rev_seq:gggcaaatactcggatt; mol_type = other DNA; note = Primer pEA29B
gggcaaatactcggatt